(19) United States
(12) Patent Application Publication
(10) Pub. No.: US 2024/0316023 A1
(43) Pub. Date: Sep. 26, 2024

(54) BENEDIN, PIPERIDINE, 2-BENZHYDRYL-3-HYDROXY-N-METHYL-, HYDROCHLORIDE AND DERIVATIVES THEREOF FOR USE IN TREATING KLEINE-LEVIN SYNDROME

(71) Applicant: NLS Pharmaceutics AG, Zurich (CH)

(72) Inventor: Eric KONOFAL, Senlis (FR)

(73) Assignee: NLS Pharmaceutics AG, Zurich (CH)

(21) Appl. No.: 18/576,918

(22) PCT Filed: Jul. 8, 2022
(86) PCT No.: PCT/EP2022/069188

§ 371 Date: Jan. 5, 2024

(30) Foreign Application Priority Data
Jul. 8, 2021 (EP) 21305946.2

(51) Int. Cl. A61K 31/445

(52) U.S. Cl. CPC A61K 31/445

(57) ABSTRACT

The invention concerns a compound of formula (I)

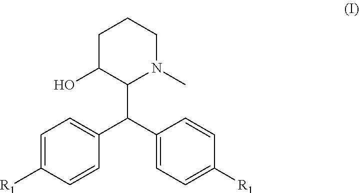

R1=H or halogen atom selected in the group consisting of: F, Cl, Br, I, or a pharmaceutically acceptable isomer, salt and/or solvate thereof, for use in preventing and/or treating Kleine-Levin syndrome.

### FIELD OF THE PRESENT INVENTION

The present invention relates to the use of Benedin, Piperidine, 2-benzhydryl-3-hydroxy-N-methyl-, hydrochloride and derivatives thereof in the prevention and/or treatment of Kleine-Levin Syndrome.

### BACKGROUND OF THE PRESENT INVENTION

The central disorders of hypersomnolence (CDH) are marked by pathologic daytime sleepiness and/or unappropriated arousal status. The International Classification of Sleep Disorders, Third Edition (ICSD-3) classifies eight different Central Disorders of Hypersomnolence: Narcolepsy Type 1, Narcolepsy Type 2, Idiopathic Hypersomnia, Kleine-Levin syndrome, hypersomnia associated with a psychiatric disorder, hypersomnia due to a medical disorder, hypersomnia due to a medication or substance, and insufficient sleep syndrome¹.

The underlying pathophysiology of these disorders is unclear.

Kleine-Levin Syndrome (KLS) is an orphan disease characterized by recurrent, relapsing-remitting episodes of severe hypersomnia, a need for excessive amounts of sleep (hypersomnolence), (i.e. for 18 to 20 hours per day): excessive food intake (compulsive hyperphagia, binge-eating); and cognitive impairment, apathy, derealization, altered mood and behavioral changes such as an abnormally uninhibited sexual drive².

Less than 500 cases with KLS have been reported in the medical literature. However, because cases of KLS often go unrecognized, the disorder is under-diagnosed, making it difficult to determine its true frequency in the general population³.

The disorder primarily affects adolescent males who appear to be affected three times as often as females and usually around the age of 16 years. When awake, affected individuals may exhibit irritability, lack of energy (lethargy), and/or lack of emotions (apathy). They may also appear confused (disoriented) and experience hallucinations with psychosis state⁴.

Symptoms of KLS are cyclical. An affected individual may go for weeks or months without experiencing symptoms. When present, symptoms may persist for days to weeks.

The exact cause of KLS is not known. However, researchers believe that in some cases, hereditary factors may cause some individuals to have a genetic predisposition of developing this disorder. It is thought that symptoms of KLS may be related to malfunction of the portion of the brain that helps to regulate functions such as sleep, appetite, and body temperature (hypothalamus)^{3,4}.

Muscarinic system plays an important role in the regulation of sleep, body temperature, feeding.

Muscarinic receptors have a well-documented role in mediating the vigilance-related effects of acetylcholine⁵⁻⁷. Activation of presynaptic muscarinic M₂ receptors promotes acetylcholine release from laterodorsal tegmental (LDT) and pedunculopontine (PPT) terminals^{5,8,9} and modulates acetylcholine release and EEG slow waves and spindles in prefrontal cortex of mice¹⁰.

Most specifically, M₂ receptors in the PPT contribute to the generation of rapid eye movement (REM) sleep^{8.11}, when muscarinic M₂ receptor antagonists block long-term REM sleep induced by specific M₂ receptor agonist (e.g. carbachol)^{5.12} and reduce cataplexyl¹³. The blockade of M₂ receptors antagonize desynchronized sleep, by increasing its latency and decreasing its percentage, decrease slow wave sleep, and enhance wakefulness^{7.14} and significantly aggravate cataplexy¹³.

A desynchronized sleep observed in KLS case reports with an important reduction in slow wave sleep (SWS) always present with progressive return to normal during the second half-period of KLS in despite of the persistence of clinical symptoms. REM sleep remains normal in the first half of the episode but decreased in the second half¹⁵.

Muscarinic system is involved in regulating body temperature^{16,17} as appetite modulation (e.g. the selective M₁ antagonist receptor scopolamine suppressing feeding need)¹⁸.

Compulsive excessive eating, behavioral changes such as an abnormally uninhibited sexual drive with schizophrenic-like mental symptoms is associated to episodic hypersomnia in KLS.

Cholinergic participation in masculine sexual behavior regulation is mediated mainly through muscarinic system¹⁹, and as the sex incidence whereby males are preponderantly if not wholly affected in KLS, muscarinic activity could be made responsible in this role also.

Muscarinic receptors present in the lumbar spinal cord induce facilitatory effects on male rat sexual behavior with an important influence on ejaculatory processes²⁰.

The participation of M₂ (and also M₃) receptors is identified to be a determining factor in the execution of these effects.

Previously, some researchers speculated that KLS may be an autoimmune disorder as NT1³.

The KLS mechanisms are still unknown, although genetics, inflammatory, and autoimmune origins have been suspected. Because KLS shares the remitting-relapsing course of multiple sclerosis and may possibly be inflammatory (at least in neuropathological cases, although inflammatory markers are absent in the cerebrospinal fluid). Lithium salts (considered as a potent anti-inflammatory drug) and intravenous steroids found partial benefits in controlled observational cohorts. On the other hand, several complex neurologic and psychiatric syndromes are now recognized as autoimmune encephalitis caused by newly identified autoantibodies²¹.

The potent role of muscarinic receptors in modulating the immune system has never been speculated as a cause of KLS, when it is reported that an activation of cholinergic system suppressed the immune system and increased the susceptibility to pathogenic infections²² and that pathogenic infection has also been noted at disease onset of KLS²³.

Antibodies against muscarinic receptors were described in autoimmune diseases including M₁ in Lambert-Eaton Myasthenic Syndrome (LEMS)²⁴ and myasthenia gravis²⁵, M₁ and M₃ Sjögren's Syndrome²⁶⁻²⁸, and M₂ in Graves' disease^{29,30}.

None antibody against muscarinic receptors has been documented in KLS.

These diseases share similarities with chronic fatigue syndrome (CFS) including fatigue and autonomic dysfunction³¹ which are unresponsive or poorly responsive to catecholaminergic stimulants (e.g. amphetamine salts).

In KLS, a lack of symptomatic and prophylactic treatments effectiveness unlike that found in treating narcolepsy and idiopathic hypersomnia symptomatology fail to offer to patients-controlled trials.

Only based on case reports and cohorts, amphetamine salts, significantly improved sleepiness but did not improve other symptoms³² as well as other stimulants (e.g. methylphenidate, modafinil) acting only on catecholaminergic systems failed to be efficient after a short symptomatic period^{33,34}. Anti-depressant drugs had no effect on preventing relapse, except in one case, in which an Monoamine Oxydase Inhibitor (moclobemide) was previously used³⁵. Anti-epileptic drugs showed, in a single case, improvement in abnormal behavior when carbamazepine was used³⁶.

Differently, lithium carbonate appears to produce a modest reduction of relapses including mild improvement of abnormal behavior (reducing the duration of episodes and decreasing relapses)³⁷⁻³⁹.

Amantadine had probably the most significant response, found to be 41% (reducing the number of episodes in patients with frequent episodes of KLS) compared to rest of stimulants^{3,40}.

Above all, it has never been speculated that another mechanism of action of amantadine or lithium salts could be explain these findings on KLS, when lithium carbonate is considered as a very strong anti-bipolar and antidepressant agent and that muscarinic M₂ receptors are genetically involved in the therapeutic response to mood disorders⁴¹.

Lithium carbonate treatment in KLS is considered as the most effective in reducing severity, but not frequency of symptomatology, but the question of the deep nature of KLS and of its relation with mood disorders is raised⁴². None complementary investigations based on potential activity of chronic lithium treatment on muscarinic system has been raised, when chronic lithium treatment reduces the activity of cholinergic neurons in cortex of mice model⁴³ and also affected the activity of cholinergic neurons in some areas of the rat brain^{44,45}, all of that suggesting that lithium may have effects on muscarinic receptors⁴⁶.

Amantadine is another agent used on KLS symptomatology. Its speculated therapeutic benefit on KLS symptomatology is still unclear, besides, is often lost in subsequent episodes³, as during the episode it-self as well as inter-episodic.

Amantadine increases dopamine synthesis and release, blocks presynaptic dopamine reuptake, and acts on NMDA receptors, but as a low-affinity uncompetitive NMDA receptor antagonist⁴⁷. Its mechanism of action on muscarinic system could explain this potent benefit has never been proposed, and above all, none other NMDA receptors antagonist tested had reported acting on KLS symptomatology.

Relaxin-3 was discovered in 2001 by searching for homologues of the relaxin gene in the Celera Discovery System and Celera Genomics databases⁴⁸ and due to its predominant expression in brain, was subsequently classified as a neuropeptide.

5 The relaxin family peptide receptor RXFP3 is the cognate receptor of relaxin-3 (also known as INSL7), a neuropeptide belonging to the insulin/relaxin superfamily. The relaxin-3/RXFP3 system is involved in the regulation of food intake, stress response, as well as arousal and exploratory behaviors including hippocampal theta rhythm and associated learning and memory⁴⁹.

In vitro, relaxin-3 can also bind and activate the relaxin-family peptide receptor RXFP1 and RXFP4 whose endogenous ligand as e.g. is DRD4 which is implicated in binge-eating, lead to obesity⁵⁰.

Polymorphisms in DRD4 gene have previously been shown to associate with a variety of behavioral phenotypes, including ADHD symptomatology, substance abuse and excessive sexual behavior⁵¹ and involved in dopaminergic tone reduction during periods of hypersomnolence in KLS⁵².

There is no evidence, publication or report based on relaxin-3 (RXFP3) and KLS when previous studies have previously suggested that the relaxin family peptide receptor 3 (relaxin/RXFP3), a G protein-coupled receptor (GPCR) was implicated in stress responses, feeding and metabolism, motivation and reward, and sexual behavior. The relaxin-3/RXFP3 system as an important “extrinsic” regulator of the neuroendocrine axis by reviewing its neuroanatomy and its putative roles in arousal-, stress-, and feeding-related behaviors and links to associated neural substrates and signaling networks⁵³⁻⁵⁵.

The relaxin-4/RXFP4 expressed in the colorectum with emerging roles in appetite regulation, during periods of calorie restriction, has a particular orexigenic role^{48,56}.

Since the human relaxin-3/ RXFP3 was found to activate relaxin-4/RXFP4 which also may be associated with obesity a potential role of RXFP3 in appetite control in patients with KLS may be potentially considered.

Current evidence identifies RXFP3 as a potential therapeutic target for treatment of neuroendocrine disorders and related behavioral dysfunction^{53,57}, but none evidence based on relaxin-3/RXFP3 system and KLS has been reported. Other findings have suggested relaxin-3/RXFP3 signaling in key hypothalamic and limbic circuits is capable of integrating stress-related external and internal information, by regulating the networks responsible for orexigenic and goal-directed (motivated) behaviors⁵⁸.

On circadian rhythm and arousal, the relaxin-3/RXFP3 signaling promote a range of consummatory behaviors is in line with its likely primary role in driving arousal and motivated behavior more broadly^{55,58,59}.

The pharmacological binding profile of Benedin has never been studied.

Benedin, Piperidine, 2-benzhydryl-3-hydroxy-N-methyl-, hydrochloride, acting as DAT and NET reuptake inhibitor, as muscarinic M1, M2 and M3 antagonist, kappa-opioid (KOP), mu-opiod (MOP) and RXFP3 partial agonist is a potent pharmacological agent interesting in the field of neurological diseases and sleep disorders, central disorders of hypersomnolence, preferably Kleine-Levin Syndrome.

### SUMMARY OF THE PRESENT INVENTION

An object of the invention is a compound of formula (I)

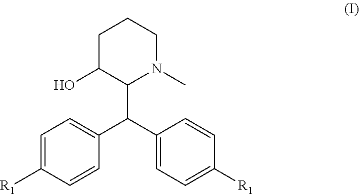

R1=H or halogen atom selected in the group consisting of: F, Cl, Br, I, or a pharmaceutically acceptable isomer, salt and/or solvate thereof, for use in preventing and/or treating Kleine-Levin Syndrome.

Another object of the invention is a pharmaceutical composition comprising a compound of formula (I) or a pharmaceutically acceptable isomer, salt and/or solvate thereof and a pharmaceutically acceptable excipient for use in preventing and/or treating Kleine-Levin Syndrome.

### FIGURES

FIG. 1 Time schedule of the test.

FIG. 2 Effects of donepezil, Benedin on the discrimination index (DI; mean±SEM and individual values). Difference vs. Control group: ns=not significant; * p≤0.05; ** p≤0.01. Difference vs. Donep 2 group: not significant in all cases except for Control group (not represented). Difference vs. 0: #p≤0.05: ###p≤0.001; Otherwise: not significant.

FIG. 3 Effects of donepezil, Benedin on the difference of exploration time between the novel object and the familiar object (N−F: mean±SEM and individual values). Difference vs. Control group: ns=not significant; *p≤0.05; ** p≤0.01. Difference vs. Donep 2 group: not significant in all cases except for Control group (not represented). Difference vs. 0: #p≤0.05; ##p≤0.01; ###p≤0.001; Otherwise: not significant.

FIG. 4 Effects of donepezil, Benedin on the exploration time during the sample trial (ST: mean±SEM and individual values). Comparisons vs. the Control group. Difference vs. Control group; ns=not significant; ** p≤0.01.

FIG. 5 Effects of donepezil, Benedin on the exploration time during the sample trial (ST: mean±SEM and individual values). Comparisons vs. the Donepezil 2 group. Difference vs. Donep 2 group: ns=not significant; * p≤0.05; ** p≤0.01; *** p≤0.001.

FIG. 6 Effects of donepezil, Benedin on the exploration time during the choice trial (CT=N+F; mean±SEM and individual values). Difference vs. Control group: ns=not significant. Difference vs. Donep 2 group: not significant in all cases (not represented).

### DETAILED DESCRIPTION OF THE PRESENT INVENTION

The first subject-matter of the invention relates to a compound of formula (I)

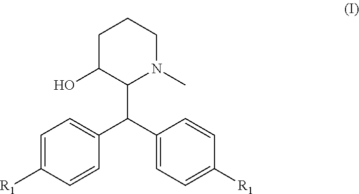

R1=H or halogen atom selected in the group consisting of: F, Cl, Br, I, preferably R1=H, or a pharmaceutically acceptable isomer, salt and/or solvate thereof, for use in preventing and/or treating Kleine-Levin syndrome.

Formula (I) has a chiral center.

Thus, “isomer” means preferably “enantiomer”.

According to the present invention, and when not specified otherwise, the term “compound of formula (I)” refers to compound of formula (I) in its racemic form or in its enantiomeric forms.

An “optically pure compound of formula (I)” means an enantiomer in an enantiomeric excess of more than 95%, preferably of more than 96%, more preferably of more than 97%, even more preferably of more than 98%, particularly preferably of more than 99%.

When R1=H, compound of formula (I) is Benedin, 2-benzhydryl-3-hydroxy-N-methyl-piperidine, a 1:1 racemic mixture and its R- and S-enantiomers, their salts, in particular their hydrochloride salt.

Compound of formula (I) is preferably used at a therapeutic dose comprised between 0.001 mg/kg/day and 0.5 mg/kg/day is administrated to a patient in need thereof, more preferably between 0.005 and 0.05 mg/kg/day.

The second subject-matter of the invention relates to a method of prevention and/or treatment of Kleine-Levin syndrome comprising the administration of a compound of formula (I) as defined above or a pharmaceutically acceptable isomer, salt and/or solvate thereof, to a patient in need thereof.

The third subject-matter of the invention relates to a pharmaceutical composition comprising a compound of formula (I) or a pharmaceutically acceptable isomer, salt and/or solvate thereof as defined above and a pharmaceutically acceptable excipient for use in preventing and/or treating Kleine-Levin syndrome.

Preferably, the pharmaceutical composition for use according to the invention comprises between 0.125 mg to 6 mg, preferably 0.25 mg to 3 mg of compound of formula (I).

Preferably, the pharmaceutical composition for use according to the invention is suitable for oral administration, for example in the form of a tablet, a capsule, a syrup, a solution, a powder or parenteral administration, for example in the form of a solution, such as an injectable solution and Transdermal Delivery Systems (TDS).

The fourth subject-matter of the invention relates to a method of prevention and/or treatment of Kleine-Levin Syndrome comprising the administration of a pharmaceutical composition as defined above to a patient in need thereof.

### EXAMPLE

### Benedin was Prepared as Shown Below, in 9 Steps.

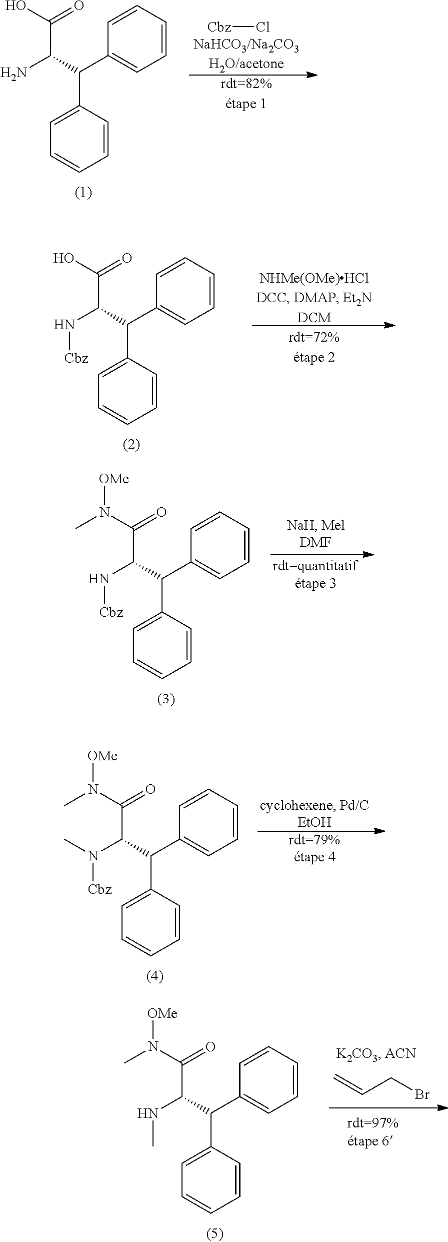

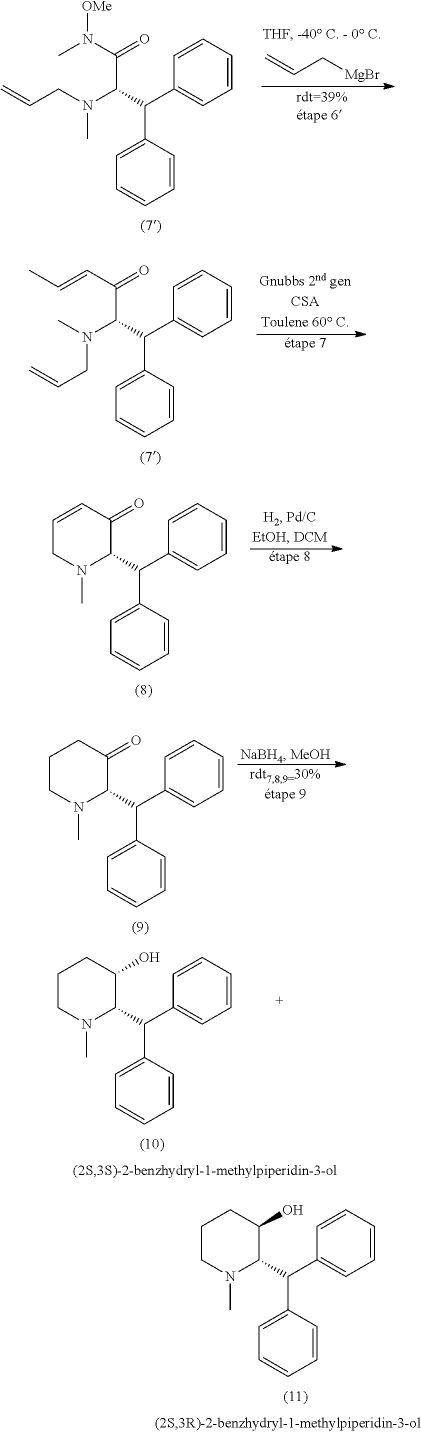

Benedin was tested at 10⁻⁵ M, calculated as a % inhibition of control specific binding of a radioactively labeled ligand specific for each target.

This binding profile panel was broadly defined with roughly an equal number of selective, central and peripheral therapeutically relevant targets, including native animal tissues, radioligands and specific enzymes involved in cell cycle regulation in accordance with Eurofins Standard Operating Procedure.

For radioligand binding experiments, the half maximal inhibitory concentration (IC₅₀) and the half maximal effective concentration (EC₅₀) values were determined (via computer software) by nonlinear regression analysis of the competition curves using Hill equation curve fitting. The inhibition constants (Kᵢ) were calculated using the Cheng-Prusoff equation (Kᵢ=IC₅₀/(1+(L/K_{D})), where L is the concentration of radioligand in the assay, and K_{D} is the affinity of the radioligand for the receptor.⁶⁰

The results are expressed as a % control specific binding ([measured specific binding/control specific binding]×100) and as a % inhibition of control specific binding (100−[(measured specific binding/control specific binding)×100] obtained in the presence of the test compounds.

Benedin was tested in a battery of additional assays for inhibition of radioligand binding by CEREP (Eurofins, France) that included human A1, A2A, and A3 adenosine receptors, α1-and α2-adrenergic receptors, human β1-adrenergic receptor, human ATI angiotensin receptor, benzodiazepine receptor, human bradykinin receptor, human CCKI cholecystokinin receptor, human D1 and D2 dopamine receptors, human endothelin receptor type A, GABAA receptor, human galactose transporter, human CXC chemokine receptors, human C-C chemokine receptor type 1, H1 and H2 histamine receptors, human MC4 melanocortin receptor, MT1 melatonin receptor, human M1, M2, and M3 muscarinic acetylcholine receptors, human NK1 and NK3 neurokinin receptors, human Y1 and Y2 neuropeptide receptors, human NTS1 neurotensin receptor, human μ-, δ-, and κ-opioid receptors and opioid-like receptor, human 5-HT1A, 5-HT1B, 5-HT2A, 5-HT3, 5-HT5A, 5-HT6, and 5-HT7 serotonin receptors, somatostatin receptor, human vasoactive intestinal peptide receptor, human vasopressin receptor, Ca2+ channel, K+v channel, SK+Ca channel, Na+ channel, and C1-channel.

Results showing an inhibition (or stimulation) lower than 25% are not considered significant and mostly attributable to variability of the signal around the control level. Low to moderate negative values have no real meaning and are attributable to variability of the signal around the control level.

An inhibition or stimulation of more than 50% is considered a significant effect of the test compounds and between 25% and 50% indicated of weak to moderate effects that should be confirmed by further testing as they are within a range where more inter-experimental variability can occur.

Fifty percent is a common cut-off for further investigation (i.e. determination of IC₅₀ or EC₅₀ values from concentration-response curves).

Principal significant or pertinent findings of these binding assays are respectively presented for Benedin in Table 1.

TABLE 1
Binding activity sites for NLS-11

| | % Inhibition at |
|---|---|
| Assay | 10⁻⁵M Benedin |
| DAT (h) (antagonist radioligand) | 99.1 |
| M₁ (h) (antagonist radioligand) | 75.0 |
| NET (h) (antagonist radioligand) | 71.9 |
| M₂ (h) (antagonist radioligand) | 65.0 |
| M₃ (h) (antagonist radioligand) | 44.6 |
| Kappa (h) (KOP) (agonist radioligand) | 39.2 |
| mu (h) (MOP) (agonist radioligand) | 24.4 |

The principal result of these binding assays confirmed that Benedin exhibited appreciable potencies for dopamine transporter (DAT) and norepinephrine transporter (NET) at 10 ⁻⁵ M concentration. Also, Benedin presented a muscarinic M1 and M2 receptors antagonist activities, which are respectively 75% and 65% at 10⁻⁵ M (Table 1). M1 more than M2 receptors antagonists have been shown to improve cognition requirement, above all M2 antagonists may be useful in treatment of behavioral disorders as well as to target cognitive impairment.

Benedin is found to weakly bind with RXFP4 and RXFP3 receptors (Study FR095-0024749-Q Eurofins/leadHunter Jun. 25, 2021; unpublished data) (Table 2).

TABLE 2
Binding activity RXFP4 and RXFP3 sites for benedin

| | % Inhibition at |
|---|---|
| Assay | 10⁻⁵M Benedin |
| RXFP4 (h) (agonist radioligand) | 26.3 |
| RXFP3 (h) (agonist radioligand) | 21.4 |

In these assays' compounds were tested in agonist and antagonist mode with the GPCR Biosensor Assays and match to this design:

### Cell Handling

1. cAMP Hunter cell lines were expanded from freezer stocks according to standard procedures.

2. Cells were seeded in a total volume of 20 μL into white walled, 384-well microplates and incubated at 37° C. for the appropriate time prior to testing.

3. cAMP modulation was determined using the DiscoverX HitHunter CAMP XS+assay.

### Gs Agonist Format

1. For agonist determination, cells were incubated with sample to induce response.

2. Media was aspirated from cells and replaced with 15 μL 2:1 HBSS/10 mM Hepes: cAMP XS+Ab reagent.

3. Intermediate dilution of sample stocks was performed to generate 4X sample in assay buffer.

4.5 μL of 4x sample was added to cells and incubated at 37° C. or room temperature for 30 or 60 minutes. Vehicle concentration was 1%.

### Gi Agonist Format

1. For agonist determination, cells were incubated with sample in the presence of EC80 forskolin to induce response.

2. Media was aspirated from cells and replaced with 15 μL 2:1 HBSS/10 mM Hepes: CAMP XS+Ab reagent.

3. Intermediate dilution of sample stocks was performed to generate 4X sample in assay buffer containing 4x EC80 forskolin.

4.5 μL of 4x sample was added to cells and incubated at 37° C. or room temperature for 30 or 60 minutes. Final assay vehicle concentration was 1%.

### Allosteric Modulation Format

1. For allosteric determination, cells were pre-incubated with sample followed by agonist induction at the EC20 concentration.

2. Media was aspirated from cells and replaced with 10 μL 1:1 HBSS/10 mM Hepes : CAMP XS+Ab reagent.

3. Intermediate dilution of sample stocks was performed to generate 4X sample in assay buffer.

4.5 μL of 4X compound was added to the cells and incubated at room temperature or 37° C. for 30 minutes.

5.5 μL of 4X EC20 agonist was added to the cells and incubated at room temperature or 37° C. for 30 or 60 minutes. For Gi-coupled GPCRs, EC80 forskolin was included.

Inverse Agonist Format (Gi only)

1. For inverse agonist determination, cells were pre-incubated with sample in the presence of EC20 forskolin.

2. Media was aspirated from cells and replaced with 15 μL 2:1 HBSS/10 mM Hepes: CAMP XS+Ab reagent.

3. Intermediate dilution of sample stocks was performed to generate 4X sample in assay buffer containing 4x EC20 forskolin.

4.5 μL of 4x sample was added to cells and incubated at 37° C. or room temperature for 30 or 60 minutes. Final assay vehicle concentration was 1%.

### Antagonist Format

1. For antagonist determination, cells were pre-incubated with sample followed by agonist challenge at the EC80 concentration.

2. Media was aspirated from cells and replaced with 10 μL 1:1 HBSS/Hepes: CAMP XS+Ab reagent.

3.5 μL of 4X compound was added to the cells and incubated at 37° C. or room temperature for 30 minutes.

4.5 μL of 4X EC80 agonist was added to cells and incubated at 37° C. or room temperature for 30 or 60 minutes. For Gi coupled GPCRs, EC80 forksolin was included.

### Signal Detection

1. After appropriate compound incubation, assay signal was generated through incubation with 20 μL cAMP XS+ED/CL lysis cocktail for one hour followed by incubation with 20 μL cAMP XS+EA reagent for three hours at room temperature.

2. Microplates were read following signal generation with a PerkinElmer Envision™ instrument for chemiluminescent signal detection.

### Data Analysis

1. Compound activity was analyzed using CBIS data analysis suite (ChemInnovation, CA).

2. For Gs agonist mode assays, percentage activity is calculated using the following formula: % Activity=100%×(mean RLU of test sample-mean RLU of vehicle control)/(mean RLU of MAX control−mean RLU of vehicle control).

3. For Gs positive allosteric mode assays, percentage modulation is calculated using the following formula: http://www.eurofinsdiscoveryservices.com Confidential Jun. 25, 2021 5% Modulation=100%×(mean RLU of test sample-mean RLU of EC20 control)/(mean RLU of MAX control−mean RLU of EC20

control).

4. For Gs antagonist or negative allosteric mode assays, percentage inhibition is calculated using the following formula: % Inhibition=100%×(1−(mean RLU of test sample-mean RLU of vehicle control)/(mean RLU of EC80 control−mean RLU of vehicle control)).

5. For Gi agonist mode assays, percentage activity is calculated using the following formula: % Activity=100%×(1−(mean RLU of test sample-mean RLU of MAX control)/(mean RLU of vehicle control−mean RLU of MAX control)).

6. For Gi positive allosteric mode assays, percentage modulation is calculated using the following formula: % Modulation=100%×(1−(mean RLU of test sample-mean RLU of MAX control)/(mean RLU of EC20 control−mean RLU of MAX control)).

7. For Gi inverse agonist mode assays, percentage activity is calculated using the following formula: % Inverse Agonist Activity=100%×((mean RLU of test sample−mean RLU of EC20 forskolin)/(mean RLU of forskolin positive control

−mean RLU of EC20 control)).

8. For Gi antagonist or negative allosteric mode assays, percentage inhibition is calculated using the following formula: % Inhibition=100%×(mean RLU of test sample−mean RLU of EC80 control)/(mean RLU of forskolin positive control−mean RLU of

EC80 control).

In these assays (Study FR095-0024749-Q Eurofins/leadHunter Jun. 25, 2021; unpublished data) compounds were tested in agonist and antagonist mode with the GPCR Biosensor Assays. For agonist and antagonist assays, data was normalized to the maximal and minimal response observed in the presence of control ligand and vehicle.

For Gi cAMP assays, the following forskolin concentration was used:
RXFP3 CAMP: 20 μM Forskolin
RXFP4 CAMP: 20 μM Forskolin

The following EC80 concentrations were used:
RXFP3 cAMP: 0.0003 μM Relaxin-3
RXFP4 CAMP: 0.01 μM Relaxin-3

The therapeutic effects on hypersomnia symptoms may be attributed to DAT and NET and we do not concede authorship for these findings but the treatment of KLS symptomatology cannot be limited to this mechanism of action of catacholamine.

We claim the paternity for implicating of muscarinic system in the pathophysiology of KLS and the effect of muscarinic receptors in the treatment of KLS.

We speculate that full or partial muscarinic receptor antagonists may be useful and effective in treating KLS symptomatology and especially as:

M₁ receptor antagonists: scopolamine, propiverine, benztropine, biperiden, pirenzepine, telenzepine, VU 0255035, PIPE-359:

M₂ receptor antagonists: himbacine, methoctramine, chlorpromazine, gallamine, thorazine, trimipramine, tolterodine, oxybutynin, otenzepad, ipratropium, hyoscyamine, diphenhydramine, dimethindene, dimenhydrinate, dicycloverine, atropine, AF-DX 116, AF-DX 384, AQ-RA 741; M₃ receptor antagonists: 4-DAMP, DAU 5884, J 104129.

We speculate that Benedin, Piperidine, 2-benzhydryl-3-hydroxy-N-methyl-, hydrochloride may target differently than methylphenidate and amphetamine salts and to be potentially useful in the prevention and/or treatment of neurological diseases associated with sleep disorders and/or central disorders of hypersomnolence, preferably Kleine-Levin syndrome.

Benedin, Piperidine, 2-benzhydryl-3-hydroxy-N-methyl-, hydrochloride targets on muscarinic system and preferably on M1, M2 and M3 muscarinic receptors as antagonist, which indirectly act on NMDA receptors, help to improve behavioral and cognitive symptoms as well as sexual disinhibition, compulsive feeding disorders, signs of dysautonomia and sleep alteration.

Rapid eye movement (REM) and non-REM (NREM) sleep have associated with improved memory performance when destabilization patterns of sleep impairs sleep-dependent long-term memory consolidation.

Cognition is severely impaired during episodes in 91% of patients with KLS, which ones have difficulties concentrating, 87% are lost in time and, in some cases, space^{13,62-64}. Patients with KLS are bedridden and unable to read, answer the phone, or perform homework, and finally have also partial or complete retrograde amnesia of events during episodes^{13,62-64}.

Several functional brain imaging studies highlighted abnormal perfusion in patients with KLS, even during asymptomatic periods^{4,65}, confirming that cognitive impairment and abnormal brain functioning during asymptomatic periods may be more frequent than expected by simple patient interviews⁶⁵. As many as 70% of patients with KLS had hypometabolism, mostly affecting the posterior associative cortex and the hippocampus⁴.

Remote novel object recognition memory has commonly associated with spindle activity during post-encoding slow-wave sleep, consistent with the view that neuronal memory replay during slow-wave sleep contributes to long-term memory formation⁶⁶⁻⁶⁸. Increasingly results indicate that the hippocampus has an important role in long-term consolidation during sleep even for memories that have previously been considered hippocampus-independent⁶⁷.

According recent findings showing a correlation of long-term NOR performance with post-encoding sleep spindle activity⁶⁷, we speculate that NOR could be helping to test Benedin and improve cognitive impairment in patients with KLS symptomatology.

### Effects of Benedin and Donepezil on Long-Term Memory in the Novel Object Recognition (NOR) Test in Mice.

The novel object recognition (NOR) test has been used in numerous studies to evaluate long-term episodic memory in rodents⁶⁹⁻⁷⁵. Donepezil, one of the most used compound for treatment of mild cognitive alteration⁷⁶, compared to memantine (close to amantadine) has been shown to improve long-term episodic memory in rats or mice^{70,72,75}. Donepezil has been clinically tested on somnolence and narcolepsy^{77,78} but none on KLS symptomatology.

The aim of this study was to examine whether Benedin improve long-term episodic memory in mice. Donepezil was used as positive control drug.

### SUMMARY

### Method

The effects on memory of Benedin (0.1, 0.5, 1 mg/kg) were compared to those of vehicle and of donepezil (2 mg/kg). Long-term episodic memory was tested in the NOR test, using a 3-days interval between the acquisition session (called sample trial) and the retention session (called choice trial). This method allows to detect an improvement of memory in the natural forgetting condition:

### Results

The Control group did not recognize the familiar object. Donepezil (2 mg/kg) improved the recognition of the familiar object, i.e. improved memory. Therefore, experimental conditions were suitable to detect an improvement of memory.

Donepezil (2 mg/kg) also decreased the exploration time 30 min post-treatment, but not 3 days post treatment.

Benedin improved the recognition of the familiar object, i.e. improved memory. This effect was significant at 0.5 mg/kg and was not significantly different to that of donepezil (2 mg/kg). It was not significant at 0.1 and 1 mg/kg.

Benedin (0.1, 0.5, 1 mg/kg) did not significantly modify the exploration time 30 min post- treatment-contrary to donepezil (2 mg/kg)-and 3 days post treatment.

### Conclusion

The results of this study suggest that benedin induced a long term memory improvement which was significant and of the same extent to that induced by donepezil. At doses tested, Benedin did not reduce exploratory behavior, contrary to donepezil, suggesting that Benedin may induce less side effect than donepezil.

### Materials and methods

General points

Manipulations of animals were conducted carefully in order to reduce stress at the minimum. All the experiments were performed in compliance with the guidelines of the French Ministry of Agriculture for experiments with laboratory animals (law 2013-118).

Experiments were conducted in standard conditions (T°=22.0±1.5° C.), with artificial light in quiet conditions (no noise except those generated by ventilation and by the apparatus used for experiments).

Experiments were conducted blindly.

The animals have not been subjected to other experiments before the study.

Animals

| | |
|---|---|
| Specie, strain, sex | Mice, C57BL/6J, male |
| Age | 6-7 weeks old at the time of testing |
| Number of animals | N = 128 |
| Origin (breeder) | Janvier Labs, France |
| Housing | Group housing (4 mice/cage): 1290D Eurostandard Type III |
| | cages (Tecniplast, Italy) in transparent polycarbonate (42.5 cm |
| | deep; 26.6 cm large; 15.5 cm high, area = 820 cm²). |
| | Cages are covered with a stainless steel grid in which food and |
| | a bottle are placed. A stainless steel removable divider |
| | separates food and water. |
| Litter | Aspen Small (SDS Dietex, France) |
| Enrichment | Wood brick, cell huts |
| Temperature | 22.0 ± 1.5° C. |
| Hygrometry | 50 ± 30% (not guaranteed; measured not controlled) |
| Air renewal | Fresh air, 12-25 vol/h |
| Lighting | 20-30 Lux |
| Day/night cycle | 12 h/12 h cycle; light on 8:00-20:00/off: 20:00-8:00 |
| Food | Rat-mouse A04 (Safe, France) available ad libitum |
| Drink | Tap water, available ad libitum |

Drugs

| | |
|---|---|
| NLS-11 | Benedin |
| Vehicle | 1% CMC + 0.5% Tween 80 in 0.9% NaCl |
| Administration route | Intraperitoneal (i.p.) |
| Doses studied | 0.1, 0.5, 1 mg/kg |
| Molecular weight base/salt | NT/NT |
| Correction factor | 1 |
| Number of applications | 1 |
| Application volume | 10 ml/kg of body weight. |
| Preparation | Stock solution aliquoted, stocked at −80° C. for 6 weeks |
| Storage conditions during test | Ambient temperature (22-23° C.) |

| | |
|---|---|
| Donepezil | Donepezil hydrochloride; C₂₄ H₃₀ Cl NO₃; CAS: |
| | 120011-70-3 |
| Supplier; Ref; Batch | Interchim; Ref AG509; Batch 05B61261 |
| Vehicle | 0.9% NaCl |
| Administration route | Intraperitoneal (i.p.) |
| Doses studied | 2 mg/kg |
| Molecular weight base/salt | 379.492/415.95 |
| Correction factor | 1.10 |
| Number of applications | 1 |
| Application volume | 10 ml/kg of body weight. |
| Preparation | Stock solution aliquoted, stocked at −80° C. for 6 weeks. |
| Storage conditions during test | Ambient temperature (22-23° C.) |

### Novel Object Recognition (NOR) Test

### Materials

The test was carried out in circular boxes (30 cm diameter, 40 cm high). The objects to be discriminated (L≈1≈h≈3-4 cm) differed in both color and shape and were referred as yellow duck and blue lego. They were fixed with a magnet to the floor of the boxes at 5 cm of the wall, 20 cm distant. Apparently, they have no natural significance for mice and they have never been associated with a reinforcement. In order to rule out the possibility of scent traces left on the objects and therefore the dependency of the recognition capacity of mice on the olfactory cue, the objects and the ground of the box were washed with an odorless disinfectant (Sanicid® diluted in water) and dried between each trial. A camcorder was fixed to the ceiling above the box to record the animals' activity. The experiment was analyzed blindly at a later time.

### Procedure

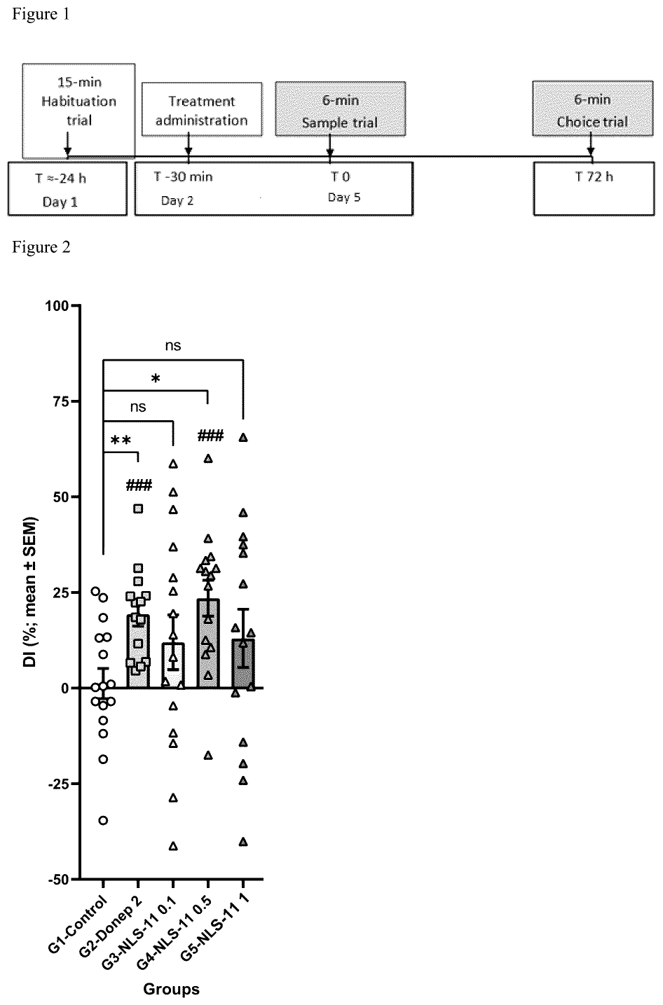

On the week before the test, animals were handled by the experimenter in charge of the experiment in order to be not stressed at the time of testing. For this purpose, the experimenter placed a small amount of litter, and then the mouse on its hand. Handling took about 1-2 min and was made every day for 4 or 5 consecutive days, and until the mouse did not show any fear to manipulations. The NOR test was completed over five days (see FIG. 1):
Day 1: Habituation trial. Animals were individually placed for a 15-min free exploration session in empty open boxes.
Day 2. Treatment administration and sample trial. The mice were administered with treatments to which they have been assigned. They were individually placed 30 min after in the apparatus for a 6-min session with two identical objects (Duck, 50% of animals or Lego, 50% of animals).
Day 3. Choice trial. The mice were individually placed for a 6-min session in the apparatus with two objects (Duck and Lego), one of the objects presented in sample trials (termed as familiar objects) and a novel object (Lego, 50% of animals or Duck, 50% of animals).

The sample and choice trials were recorded with the camera located above the apparatus. The time spent by mice in exploring the objects was measured during the sample trial and the choice trial.

Exploration of an object was defined as follows: directing the nose to the object at a distance≤2 cm and/or touching it with the nose or forelimbs: turning around or biting the object, or sitting on the object were not considered as exploratory behavior.

### Read-Outs

Data recorded:
L=exploration time of the left object in the sample trial.
R=exploration time of the right object in the sample trial.
N=exploration time of the new object in the choice trial.
F=exploration time of the familiar object in the choice trial.
   Object recognition task indices include the following parameters:
Exploration indices:
   ST=L+R=exploration time (left object+right object) in the sample trial.
   CT=N+F=exploration time (new object+familiar object) in the choice trial.
Two memory indices:
   N−F=difference of exploration time between the new object and the familiar object in the choice trial.

$$DI = discrimination index = 100 \times ( N - F ) / ( N + F ) .$$

### Groups

Animals (N=80) were divided into 5 groups (N=16/ group) which received 30 min before the sample trial an injection of:
G1-Control group: Vehicle
G2-Donep 2 group: Donepezil (2 mg/kg)
G3-Benedin 0.1 group: Benedin (0.1 mg/kg)
G4-Benedin 0.5 group: Benedin (0.5 mg/kg)
G5-Benedin 1 group: Benedin (1 mg/kg)
   Data analysis

Statistical analyses were performed using the GraphPad Prism 9 software.

Data are expressed as mean and standard error of mean (SEM).

A difference is considered statistically significant at p≤0.05.

Statistical analyses:
Read-outs: DI, N−F, ST, CT, N, F
   Unpaired Student's t test: Donep 2 group vs. Control group.
   One-way ANOVA, Dunnett test:
      Benedin groups vs. Control group.
      Benedin groups vs. Donep 2 group.
Read-outs: DI and N−F, for all groups, paired Student's t test, difference vs. 0.
Body weight: one-way ANOVA.

Exclusion criteria: animals which displayed a poor exploratory behavior, i.e. which spent less than 5 sec in exploring the two objects in the sample trial and/or in the choice trial were discarded from the analysis for DI and N−F. All animals were included in the analysis for ST and CT

### Results

Only the most meaningful results, i.e. the effects of treatments on the memory indices (discrimination index and the difference of exploration time between the novel object and the familiar object) and on the exploration indices (exploration time during the sample and choice trials) are described below.

The body weight was not significantly different between groups (ANOVA: F(7:120)=0.9303: p=0.486; see Table 6).

Control group, effect of donepezil

The results are presented in Table 3.

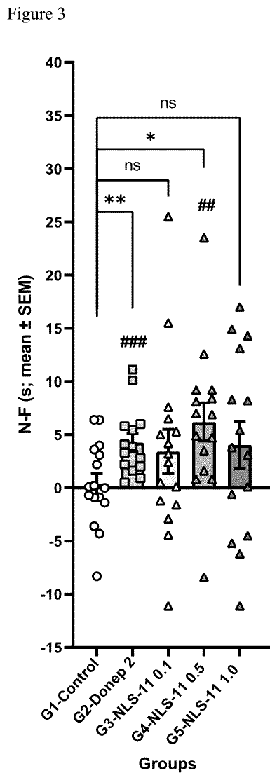

The discrimination index (DI, FIG. 2): and the difference of exploration time between the novel object and the familiar object (N−F: FIG. 3) were not significantly higher than zero for the ontrol group.

The discrimination index (DI, FIG. 2) and the difference of exploration time between the novel object and the familiar object (N−F: FIG. 3) were significantly higher than zero. Both the DI and the ‘N−F’ were significantly higher in the Donep 2 group than in the Control group.

Summary. The Control group did not recognize the familiar object. Donepezil (2 mg/kg) improved the recognition of the familiar object, i.e. improved memory. Therefore, experimental conditions were suitable to detect an improvement of memory.

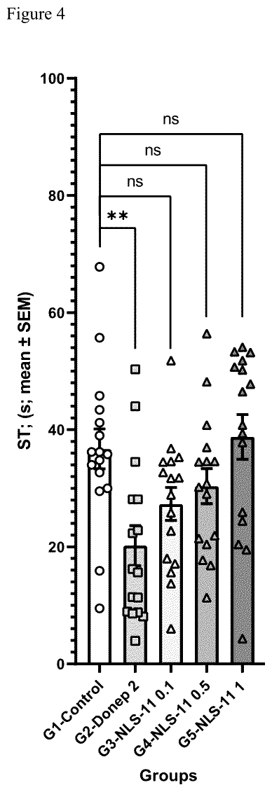

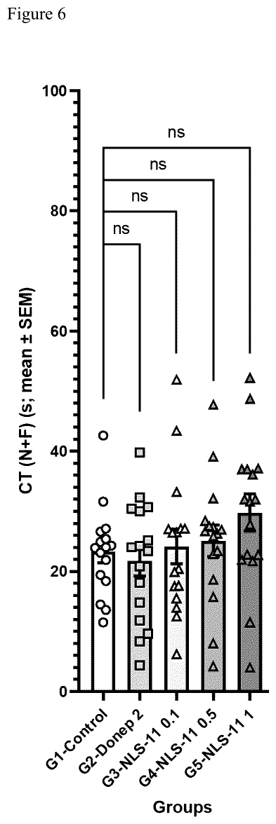

Donepezil (2 mg/kg) decreased the exploration time during the sample trial (ST: 30 min post treatment; FIG. 4) and did not significantly modify the exploration time during the choice trial (CT; 72 h post treatment; FIG. 6).

Summary, Donepezil (2 mg/kg) decreased the exploration time 30 min post-treatment, but not 3 days post treatment.

TABLE 3
Control and Donep 2 groups. Indices of memory: discrimination index (DI) and difference
of exploration time between the novel object and the familiar object (N − F). Indices
of exploration: exploration time during the sample trial (ST) and during the choice trial
(CT). Results are expressed as mean and SEM. Statistical analyses: “p vs. random”, difference
vs. 0 (paired Student's t test); “p vs. G1-Control”, unpaired Student's t test.

| | | | N − F | ST | CT |
|---|---|---|---|---|---|
| | | DI | Exploration time | Exploration | Exploration time |
| | | Discrimination Index = | novel − familiar | time in the | in the choice |
| Group | | 100 × (N − F)/(N + F) | object (s) | sample trial (s) | trial (s) = N + F |
| G1-Control | Mean | 1.2 | 0.4 | 36.7 | 23.3 |
| N = 16 | SEM | 3.9 | 1.0 | 3.4 | 1.9 |
| p vs. Random≤ | | 0.767 | 0.703 | | |
| G2-Donep 2 | Mean | 19.3 | 4.3 | 20.2 | 21.7 |
| N = 16 | SEM | 3.2 | 0.9 | 3.4 | 2.5 |

| | | | | |
|---|---|---|---|---|
| p vs. Random≤ | 0.001 | 0.001 | | |
| p vs. G1-Control≤ | 0.002 | 0.007 | 0.002 | 0.616 |

### Effect of Benedin

The results are presented in Table 4.

The discrimination index (DI, FIG. 2):
For the Benedin 0.1 group: was not significantly different from 0 and was not significantly different from that of the control group and from that of the Donep 2 group.
For the Benedin 0.5 group: was significantly higher than 0, significantly higher than that of the control group and was not significantly different from that of the Donep 2 group.
For the Benedin 1 group: was not significantly different from 0 and was not significantly different from that of the control group and from that of the Donep 2 group.
   The difference of exploration time between the novel object and the familiar object (N−F; FIG. 3):
For the Benedin 0.1 group: was not significantly different from 0 and was not significantly different from that of the control group and from that of the Donep 2 group.
For the Benedin 0.5 group: was significantly higher than 0, tended to be higher than that of the control group and was not significantly different from that of the Donep 2 group.
For the Benedin 1 group: was not significantly different from 0 and was not significantly different from that of the control group and from that of the Donep 2 group.

Summary. Benedin improved the recognition of the familiar object, i.e. improved memory. This effect was significant at 0.5 mg/kg and was not significantly different to that of donepezil (2 mg/kg).

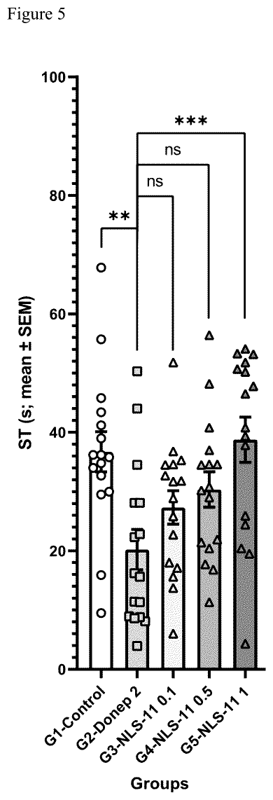

The exploration time during the sample trial (ST; 30 min post treatment):
For the Benedin 0.1 group: was not significantly different from that of the control group (FIG. 4) and was not significantly different from that of the Donep 2 group (FIG. 5).
For the Benedin 0.5 group: was not significantly different from that of the control group (FIG. 4) and tended to be higher than that of the Donep 2 group (FIG. 5).
For the Benedin 1 group: was not significantly different from that of the control group (FIG. 4) and was significantly higher than that of the Donep 2 group (FIG. 5).

For Benedin 0.1, 0.5 and 1 groups, the exploration time during the choice trial (CT; 72 h post treatment; FIG. 6): was not significantly different from that of the control group and was not significantly different from that of the Donep 2 group.

Summary. Benedin (0.1, 0.5, 1 mg/kg) did not significantly modify the exploration time 30 min post-treatment-contrary to donepezil (2 mg/kg)-and 3 days post treatment.

TABLE 4
Control and Benedin 0.1, 0.5 and 1 groups. Indices of memory: discrimination index (DI) and difference
of exploration time between the novel object and the familiar object (N − F). Indices of
exploration: exploration time during the sample trial (ST) and during the choice trial (CT).
Results are expressed as mean and SEM. Statistical analyses: “p vs. random”, difference
vs. 0 (paired Student's t test; one-way ANOVAs for comparisons Benedin (NLS-11) and Control
groups and for comparisons Benedin (NLS-11) and Donep 2 groups; “p vs. G1-Control”, Dunnett's
test (except Donep 2 group: unpaired Student's t test); “p vs. G2-Donep 2”, Dunnett's test.

| | | | N − F | ST | CT |
|---|---|---|---|---|---|
| | | DI | Exploration time | Exploration | Exploration time |
| | | Discrimination Index = | novel − familiar | time in the | in the choice |
| Group | | 100 × (N − F)/(N + F) | object (s) | sample trial (s) | trial (s) = N + F |
| G1-Control | Mean | 1.2 | 0.4 | 36.7 | 23.3 |
| N = 16 | SEM | 3.9 | 1.0 | 3.4 | 1.9 |
| p vs. Random≤ | | 0.767 | 0.703 | | |
| G2-Donep 2 | Mean | 19.3 | 4.3 | 20.2 | 21.7 |
| N = 16 | SEM | 3.2 | 0.9 | 3.4 | 2.5 |
| p vs. Random≤ | | 0.001 | 0.001 | | |
| p vs. G1-Control≤ | | 0.002 | 0.007 | 0.002 | 0.616 |
| G3-NLS-11 0.1 | Mean | 12.0 | 3.4 | 27.3 | 24.1 |
| N = 16 | SEM | 7.2 | 2.1 | 2.8 | 2.9 |
| p vs. Random≤ | | 0.116 | 0.125 | | |
| p vs. G1-Control≤ | | 0.443 | 0.494 | 0.119 | 0.993 |
| p vs. G2-Donep 2≤ | | 0.723 | 0.977 | 0.299 | 0.875 |
| G4-NLS-11 0.5 | Mean | 23.5 | 6.2 | 30.4 | 25.0 |
| N = 16 | SEM | 4.7 | 1.8 | 3.0 | 2.6 |
| p vs. Random≤ | | 0.001 | 0.004 | | |
| p vs. G1-Control≤ | | 0.032 | 0.073 | 0.386 | 0.939 |
| p vs. G2-Donep 2≤ | | 0.933 | 0.809 | 0.084 | 0.737 |
| G5-NLS-11 1 | Mean | 13.0 | 4.0 | 38.8 | 29.8 |
| N = 16 | SEM | 7.6 | 2.2 | 3.8 | 3.1 |
| p vs. Random≤ | | 0.112 | 0.093 | | |
| p vs. G1-Control≤ | | 0.385 | 0.358 | 0.948 | 0.217 |
| p vs. G2-Donep 2≤ | | 0.805 | 1.000 | 0.001 | 0.116 |
| ANOVA | dF | 3; 58 | 3; 58 | 3; 60 | 3; 60 |
| Control and NLS- | F | 2.263 | 1.745 | 2.653 | 1.178 |
| 11 groups | p | 0.091 | 0.168 | 0.057 | 0.326 |
| ANOVA | dF | 3; 56 | 3; 56 | 3; 60 | 3; 60 |
| Donep 2 and NLS- | F | 0.8127 | 0.4226 | 5.459 | 1.459 |
| 11 groups | p | 0.493 | 0.168 | 0.003 | 0.235 |

Additional analyses

TABLE 6
Body weight (BW), exploration time of the novel object (N) and exploration time of
the familiar object (F). Statistical analyses: “p vs. random”, difference vs.
N vs. F (paired Student's t test; “p vs. G1-Control”; other analyses; see Table 3-Table 5.

| | | | N | F |
|---|---|---|---|---|
| | | BW | Exploration time of | Exploration time of |
| Group | | Body weight (g) | novel object (s) | familiar object (s) |
| G1-Control | Mean | 22.5 | 11.8 | 11.5 |
| N = 16 | SEM | 0.4 | 1.0 | 1.1 |
| p vs. Random≤ | | | 0.703 | |
| G2-Donep 2 | Mean | 23.2 | 13.1 | 8.7 |
| N = 16 | SEM | 0.3 | 1.6 | 1.0 |
| p vs. Random≤ | | 0.510 | 0.001 | |
| p vs. G1-Control≤ | | | 0.529 | 0.058 |
| G3-NLS-11 0.1 | Mean | 22.9 | 13.8 | 10.4 |
| N = 16 | SEM | 0.4 | 2.1 | 1.3 |
| p vs. Random≤ | | 0.904 | 0.125 | |
| p vs. G1-Control≤ | | | 0.790 | 0.897 |
| p vs. G2-Donep 2≤ | | | 0.989 | 0.706 |
| G4-NLS-11 0.5 | Mean | 22.8 | 15.4 | 9.6 |
| N = 16 | SEM | 0.3 | 1.7 | 1.4 |
| p vs. Random≤ | | 0.981 | 0.004 | |
| p vs. G1-Control≤ | | | 0.359 | 0.659 |
| p vs. G2-Donep 2≤ | | | 0.714 | 0.928 |
| G5-NLS-11 1 | Mean | 23.1 | 16.8 | 13.0 |
| N = 16 | SEM | 0.4 | 2.0 | 1.7 |
| p vs. Random≤ | | 0.725 | 0.091 | |
| p vs. G1-Control≤ | | | 0.140 | 0.785 |
| p vs. G2-Donep 2≤ | | | 0.384 | 0.075 |
| ANOVA | df | | 3; 60 | 3; 60 |
| Control and NLS- | F | | 1.415 | 1.118 |
| 11 groups | p | | 0.248 | 0.350 |
| ANOVA | df | | 3; 60 | 3; 60 |
| Donep 2 and NLS- | F | | 0.768 | 1.842 |
| 11 groups | p | | 0.517 | 0.150 |

### Bibliography

1. American Academy of Sleep Medicine. International Classification of Sleep Disorders. American Acad. of Sleep Medicine; 2014.

2. Trotti L M, Arnulf I. Idiopathic Hypersomnia and Other Hypersomnia Syndromes. Neurotherapeutics. Published online Sep. 8, 2020. doi:10.1007/s13311-020-00919-1

3. Arnulf I. Kleine-Levin Syndrome. Sleep Med Clin. 2015;10(2):151-161. doi: 10.1016/j.jsmc.2015.02.001

4. Dudoignon B, Tainturier L E, Dodet P, et al. Functional brain imaging using 18F-fluorodeoxyglucose positron emission tomography/computerized tomography in 138 patients with Kleine-Levin syndrome: an early marker? Brain Commun. 2021;3(2):fcab130. doi:10.1093/braincomms/fcab130

5. Baghdoyan H A, Lydic R. M2 muscarinic receptor subtype in the feline medial pontine reticular formation modulates the amount of rapid eye movement sleep. Sleep. 1999;22(7):835-847. doi:10.1093/sleep/22.7.835

Brischoux F. Mainville L. Jones B E. Muscarinic-2 and orexin-2 receptors on GABAergic and other neurons in the rat mesopontine tegmentum and their potential role in sleep-wake state control. J Comp Neurol. 2008;510(6):607-630. doi:10.1002/cne.21803

7. Turner J, Hughes L F, Toth L A. Sleep, activity, temperature and arousal responses of mice deficient for muscarinic receptor M2 or M4. Life Sci. 2010;86(5-6):158-169. doi:10.1016/j.lfs.2009.11.019

8. Coleman C G, Lydic R, Baghdoyan H A. M2 muscarinic receptors in pontine reticular formation of C57BL/6J mouse contribute to rapid eye movement sleep generation. Neuroscience. 2004;126(4):821-830. doi:10.1016/j.neuroscience.2004.04.029

9. Stenberg D. Neuroanatomy and neurochemistry of sleep. Cell Mol Life Sci. 2007;64(10):1187-1204. doi:10.1007/s00018-007-6530-3

10. Douglas C L, Baghdoyan H A, Lydic R. Prefrontal cortex acetylcholine release, EEG slow waves, and spindles are modulated by M2 autoreceptors in C57BL/6J mouse. J Neurophysiol. 2002;87(6):2817-2822. doi:10.1152/jn.2002.87.6.2817

11. Yeomans J S. Muscarinic receptors in brain stem and mesopontine cholinergic arousal functions. Handb Exp Pharmacol. 2012;(208):243-259. doi:10.1007/978-3-642-23274-9_11

12. Datta S, Quattrochi J J, Hobson J A. Effect of specific muscarinic M2 receptor antagonist on carbachol induced long-term REM sleep. Sleep. 1993;16(1):8-14.

13. Arnulf I, Lecendreux M, Franco P, Dauvilliers Y. Le syndrome de Kleine-Levin. Revue Neurologique. 2008;164(8-9):658-668. doi:10.1016/j.neurol.2008.04.020

14. Imeri L, Bianchi S, Angeli P, Mancia M. Selective blockade of different brain stem muscarinic receptor subtypes: effects on the sleep-wake cycle. Brain Res. 1994;636(1):68-72. doi:10.1016/0006-8993(94)90176-7

15. Huang Y S, Lin Y H, Guilleminault C. Polysomnography in Kleine-Levin syndrome. Neurology. 2008;70(10):795-801. doi:10.1212/01.wnl.0000304133.00875.2b

16. Ghanta V K, Demissie S, Hiramoto N S, Hiramoto R N. Conditioning of body temperature and natural killer cell activity with arecoline, a muscarinic cholinergic agonist. Neuroimmunomodulation. 1996;3(4):233-238. doi:10.1159/000097276

17. Calogero A E, Kamilaris T C, Gomez M T, et al. The muscarinic cholinergic agonist arecoline stimulates the rat hypothalamic-pituitary-adrenal axis through a centrally-mediated corticotropin-releasing hormone-dependent mechanism. Endocrinology. 1989;125(5):2445-2453. doi:10.1210/endo-125-5-2445

18. Perry M L, Pratt W E, Baldo B A. Overlapping striatal sites mediate scopolamine-induced feeding suppression and mu-opioid-mediated hyperphagia in the rat. Psychopharmacology (Berl). 2014;231(5):919-928. doi:10.1007/s00213-013-3317-0

19. Retana-Marquez S, Salazar E D, Velazquez-Moctezuma J. Muscarinic and nicotinic influences on masculine sexual behavior in rats: effects of oxotremorine, scopolamine, and nicotine. Pharmacol Biochem Behav. 1993;44(4):913-917. doi:10.1016/0091-3057(93)90024-n

20. Gómez-Martinez L E, Cueva-Rolón R. Muscarinic receptor antagonism at the spinal cord level causes inhibitory effects on male rat sexual behavior. Behav Brain Res. 2009;203(2):247-255. doi:10.1016/j.bbr.2009.05.010

21. Arnulf I, Groos E, Dodet P. Speculating on Kleine-Levin Syndrome mechanisms. J Clin Sleep Med. 2021;17(3):611-612. doi:10.5664/jcsm.9104

22. Razani-Boroujerdi S, Behl M, Hahn F F, Pena-Philippides J C, Hutt J, Sopori M L. Role of muscarinic receptors in the regulation of immune and inflammatory responses. Journal of Neuroimmunology. 2008;194(1):83-88. doi: 10.1016/j.jneuroim.2007.11.019

23. Merriam A E. Kleine-Levin syndrome following acute viral encephalitis. Biol Psychiatry. 1986;21(13):1301-1304. doi:10.1016/0006-3223(86)90313-6

24. Suzuki S. Lambert-Eaton myasthenic syndrome (LEMS). Brain and nerve=Shinkei kenkyu no shinpo. 2010;62(4):419-426.

25. Takamori M, Motomura M, Fukudome T, Yoshikawa H. Autoantibodies against M1 muscarinic acetylcholine receptor in myasthenic disorders. European journal of neurology. 2007;14(11):1230-1235.

26. Abe S, Tsuboi H, Kudo H, et al. M3 muscarinic acetylcholine receptor-reactive Th17 cells in primary Sjögren's syndrome. JCI Insight. 2020;5(15):135982. doi:10.1172/jci.insight.135982

27. Schegg V, Vogel M, Didichenko S, et al. Evidence that anti-muscarinic antibodies in Sjögren's syndrome recognise both M3R and M1R. Biologicals. 2008;36(4):213-222.

28. Sumida T, Tsuboi H, Iizuka M, Asashima H, Matsumoto I. Anti-M3 muscarinic acetylcholine receptor antibodies in patients with Sjögren's syndrome. Modern rheumatology. 2013;23(5):841-845.

29 Galloway A, Li H, Vanderlinde-Wood M, et al. Activating autoantibodies to the β1/2-adrenergic and M2 muscarinic receptors associate with atrial tachyarrhythmias in patients with hyperthyroidism. Endocrine. 2015;49(2):457-463.

30. Stavrakis S, Yu X, Patterson E, et al. Activating autoantibodies to the beta-1 adrenergic and m2 muscarinic receptors facilitate atrial fibrillation in patients with Graves' hyperthyroidism. Journal of the American College of Cardiology. 2009;54(14):1309-1316.

31. Halder N, Lal G. Cholinergic System and Its Therapeutic Importance in Inflammation and Autoimmunity. Front Immunol. 2021;12:660342. doi:10.3389/fimmu.2021.660342

32. EARLE BV. PERIODIC HYPERSOMNIA AND MEGAPHAGIA (THE KLEINE-LEVIN SYNDROME). Psychiatr Q. 1965;39:79-83. doi: 10.1007/BF01569450

33. Rezvanian E, Watson N F. Kleine-levin syndrome treated with clarithromycin. J Clin Sleep Med. 2013;9(11):1211-1212. doi:10.5664/jcsm.3176

34. de Oliveira M M, Conti C, Prado G F. Pharmacological treatment for Kleine-Levin syndrome. Cochrane Database Syst Rev. 2016;2016(5):CD006685. doi:10.1002/14651858.CD006685. pub4

35. Chaudhry H R. Clinical use of moclobemide in Kleine-Levin syndrome. Br J Psychiatry. 1992;161:720. doi: 10.1192/bjp.161.5.720a

36. Mukaddes N M, Kora M E, Bilge S. Carbamazepine for Kleine-Levin syndrome. J Am Acad Child Adolesc Psychiatry. 1999;38(7):791-792. doi:10.1097/00004583-199907000-00001

37. Poppe M, Friebel D, Reuner U, Todt H, Koch R, Heubner G. The Kleine-Levin syndrome—effects of treatment with lithium—. Neuropediatrics. 2003;34(3):113-119. doi:10.1055/s-2003-41273

38. Smolik P, Roth B. [Diagnosis, etiopathogenesis and treatment of the Kleine-Levin syndrome]. Cesk Psychiatr. 1986;82(2):127-130.

39. Abe K. Lithium prophylaxis of periodic hypersomnia. Br J Psychiatry. 1977;130:312-313. doi: 10.1192/bjp.130.3.312

40. Ortega-Albás J J, López R, Martinez A, Carratalá S, Echeverria I, Ortega P. Kleine-Levin syndrome, GABA, and glutamate. J Clin Sleep Med. 2021;17(3):609-610. doi:10.5664/jcsm.9058

41. Gibbons AS, Jeon W J, Scarr E, Dean B. Changes in Muscarinic M2 Receptor Levels in the Cortex of Subjects with Bipolar Disorder and Major Depressive Disorder and in Rats after Treatment with Mood Stabilisers and Antidepressants. Int J Neuropsychopharmacol. 2016;19(4):pyv118. doi: 10.1093/ijnp/pyv118

42. Lemire I. Revue du syndrome de Kleine-Levin: vers une approche intégrée. The Canadian Journal of Psychiatry. 1993;38(4):277-284.

43. Miyauchi T, Oikawa S, Kitada Y. Effects of lithium chloride on the cholinergic system in different brain regions in mice. Biochem Pharmacol. 1980;29(4):654-657. doi:10.1016/0006-2952(80)90393-7

44. Jope R S. Lithium selectively potentiates cholinergic activity in rat brain. Prog Brain Res. 1993;98:317-322. doi: 10.1016/s0079-6123(08)62414-0

45. Jope R S. High affinity choline transport and acetylCoA production in brain and their roles in the regulation of acetylcholine synthesis. Brain Res. 1979;180(3):313-344. doi:10.1016/0165-0173(79)90009-2

46. Hruska R E, Ludmer L M, Pert A, Bunney W E J. Effects of lithium on [3H](-)quinuclidinyl benzilate [(3H](-)QNB) binding to rat brain muscarinic cholinergic receptors. J Neurosci Res. 1984;11(2):171-177. doi:10.1002/jnr.490110206

47. Kornhuber J, Weller M, Schoppmeyer K, Riederer P. Amantadine and memantine are NMDA receptor antagonists with neuroprotective properties. J Neural Transm Suppl. 1994;43:91-104.

48. Bathgate RAD, Halls M L, van der Westhuizen E T, Callander G E, Kocan M, Summers R J. Relaxin family peptides and their receptors. Physiol Rev. 2013:93(1):405-480. doi:10.1152/physrev.00001.2012

49. Liu C, Chen J, Sutton S, et al. Identification of relaxin-3/INSL7 as a ligand for GPCR142. J Biol Chem. 2003;278(50):50765-50770. doi:10.1074/jbc.M308996200

50. Barth B, Bizarro L, Miguel P M, et al. Genetically predicted gene expression of prefrontal DRD4 gene and the differential susceptibility to childhood emotional eating in response to positive environment. Appetite. 2020;148:104594. doi:10.1016/j.appet.2020.104594

51. Halley A C, Boretsky M, Puts D A, Shriver M. Self-Reported Sexual Behavioral Interests and Polymorphisms in the Dopamine Receptor D4 (DRD4) Exon III VNTR in Heterosexual Young Adults. Arch Sex Behav. 2016;45(8):2091-2100. doi:10.1007/s10508-015-0646-6

52. Chesson A L J, Levine S N, Kong L S, Lee S C. Neuroendocrine evaluation in Kleine-Levin syndrome: evidence of reduced dopaminergic tone during periods of hypersomnolence. Sleep. 1991;14(3):226-232.

53. Ganella D E, Ma S, Gundlach A L. Relaxin-3/RXFP3 Signaling and Neuroendocrine Function—A Perspective on Extrinsic Hypothalamic Control. Front Endocrinol (Lausanne). 2013;4:128. doi:10.3389/fendo.2013.00128

54. Smith C M, Shen P J, Banerjee A, et al. Distribution of relaxin-3 and RXFP3 within arousal, stress, affective, and cognitive circuits of mouse brain. J Comp Neurol. 2010;518(19):4016-4045. doi:10.1002/cne.22442

55. dé vila C, Chometton S, Ma S, et al. Effects of chronic silencing of relaxin-3 production in nucleus incertus neurons on food intake, body weight, anxiety-like behaviour and limbic brain activity in female rats. Psychopharmacology (Berl). 2020;237(4):1091-1106. doi:10.1007/s00213-019-05439-1

56. Grosse J, Heffron H, Burling K, et al. Insulin-like peptide 5 is an orexigenic gastrointestinal hormone. Proc Natl Acad Sci U S A. 2014;111(30):11133-11138. doi:10.1073/pnas. 1411413111

57. Ganella D E, Ryan P J, Bathgate R A D, Gundlach A L. Increased feeding and body weight gain in rats after acute and chronic activation of RXFP3 by relaxin-3 and receptor-selective peptides: functional and therapeutic implications. Behav Pharmacol. 2012;23(5-6):516-525. doi:10.1097/FBP.0b013e3283576999

58. Ma S, Smith C M, Blasiak A, Gundlach A L. Distribution, physiology and pharmacology of relaxin-3/RXFP3 systems in brain. Br J Pharmacol. 2017;174(10):1034-1048. doi:10.1111/bph.13659

59. Kumar J R, Rajkumar R, Jayakody T, et al. Relaxin' the brain: a case for targeting the nucleus incertus network and relaxin-3/RXFP3 system in neuropsychiatric disorders. Br J Pharmacol. 2017;174(10):1061-1076. doi:10.1111/bph. 13564

60. Cheng Y, Prusoff W H. Relationship between the inhibition constant (K1) and the concentration of inhibitor which causes 50 per cent inhibition (I50) of an enzymatic reaction. Biochem Pharmacol. 1973;22(23):3099-3108. doi:10.1016/0006-2952(73)90196-2

62. Lavault S, Golmard J L, Groos E, et al. Kleine-Levin syndrome in 120 patients: differential diagnosis and long episodes. Ann Neurol. 2015;77(3):529-540. doi:10.1002/ana.24350

63. Huang Y S, Guilleminault C, Lin K L, Hwang F M, Liu F Y, Kung Y P. Relationship between Kleine-Levin syndrome and upper respiratory infection in Taiwan. Sleep. 2012;35(1):123-129. doi:10.5665/sleep. 1600

64. Arnulf I, Rico T J, Mignot E. Diagnosis, disease course, and management of patients with Kleine-Levin syndrome. Lancet Neurol. 2012;11(10):918-928. doi:10.1016/S1474-4422(12)70187-4

65. Uguccioni G, Lavault S, Chaumereuil C, Golmard J L, Gagnon J F, Arnulf I. Long-Term Cognitive Impairment in Kleine-Levin Syndrome. Sleep. 2016;39(2):429-438. doi:10.5665/sleep.5458

66. Tononi G, Cirelli C. Sleep and the price of plasticity: from synaptic and cellular homeostasis to memory consolidation and integration. Neuron. 2014;81(1):12-34.

67. Sawangjit A, Oyanedel C N, Niethard N, Salazar C, Born J, Inostroza M. The hippocampus is crucial for forming non-hippocampal long-term memory during sleep. Nature. 2018;564(7734):109-113. doi:10.1038/s41586-018-0716-8

68. Stickgold R. Sleep-dependent memory consolidation.Nature. 2005;437(7063):1272-1278.

69. Birks J S, Harvey R J. Donepezil for dementia due to Alzheimer's disease. Cochrane Database Syst Rev. 2018:6(6):CD001190. doi:10.1002/14651858.CD001190.pub3

70. Prickaerts J, Heckman P R A, Blokland A. Investigational phosphodiesterase inhibitors in phase I and phase II clinical trials for Alzheimer's disease. null. 2017;26(9):1033-1048. doi:10.1080/13543784.2017.1364360

71. Chao O Y, Huston J P, Li J S, Wang A L, de Souza Silva M A. The medial prefrontal cortex-lateral entorhinal cortex circuit is essential for episodic-like memory and associative object-recognition. Hippocampus. 2016;26(5):633-645. doi:10.1002/hipo.22547

72. de Bruin N M W J, Prickaerts J, Lange J H M, et al. SLV330, a cannabinoid CB1 receptor antagonist, ameliorates deficits in the T-maze, object recognition and Social Recognition Tasks in rodents. Neurobiol Learn Mem. 2010;93(4):522-531. doi:10.1016/j.nlm.2010.01.010

73. Ennaceur A, Cavoy A, Costa J C, Delacour J. A new one-trial test for neurobiological studies of memory in rats. II: Effects of piracetam and pramiracetam. Behav Brain Res. 1989;33(2):197-207. doi:10.1016/s0166-4328(89)80051-8

74. Pichat P, Bergis O E, Terranova J P, et al. SSR180711, a novel selective alpha7 nicotinic receptor partial agonist: (II) efficacy in experimental models predictive of activity against cognitive symptoms of schizophrenia. Neuropsychopharmacology. 2007;32(1):17-34. doi:10.1038/sj.npp. 1301188

75. Terry A V J, Beck W D, Lin P C, Callahan P M, Rudic R D, Hamrick M W. Manganese-enhanced magnetic resonance imaging method detects age-related impairments in axonal transport in mice and attenuation of the impairments by a microtubule-stabilizing compound. Brain Res. 2022;1789:147947. doi:10.1016/j.brainres.2022.147947

76. Arvanitakis Z, Shah R C, Bennett D A. Diagnosis and Management of Dementia: Review. JAMA. 2019;322(16):1589-1599. doi:10.1001/jama.2019.4782

77. Kogure T, Sumitani M, Ikegami K, et al. Donepezil, an Acetylcholinesterase Inhibitor, Can Attenuate Gabapentinoid-Induced Somnolence in Patients with Neuropathic Pain: A Retrospective Chart Review. J Pain Palliat Care Pharmacother. 2017;31(1):4-9. doi: 10.1080/15360288.2017.1279500

78. Niederhofer H. Donepezil in the treatment of narcolepsy. J Clin Sleep Med. 2006;2(1): 71-72.

1-7. (canceled)

8. A method of prevention and/or treatment of Kleine-Levin syndrome comprising the administration of a compound of formula (I) 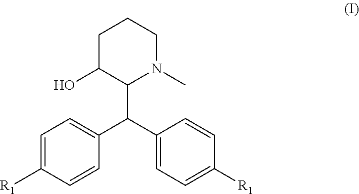
with R₁=H or a halogen atom selected from the group consisting of F, CI, Br, and I,
or a pharmaceutically acceptable isomer, salt, and/or solvate thereof, to a patient in need thereof.

9. The method of claim 8, wherein a therapeutic dose of 0.001 mg/kg/day to 5 mg/kg/day is administered to the patient in need thereof.

10. The method of claim 9, wherein the therapeutic dose is 0.005 mg/kg/day to 0.05 mg/kg/day.

11. The method of claim 8, wherein R₁=H.

12. A method of prevention and/or treatment of Kleine-Levin Syndrome comprising the administration of a pharmaceutical composition comprising a compound of formula (I) as defined in claim 8 or a pharmaceutically acceptable isomer, salt, and/or solvate thereof, and a pharmaceutically acceptable excipient.

13. The method of claim 12, wherein the pharmaceutical composition comprises 0.125 mg to 6 mg of the compound of formula (I).

14. The method of claim 12, wherein the pharmaceutical composition comprises 0.25 mg to 3 mg of the compound of formula (I).

15. The method of claim 12, wherein the pharmaceutical composition is suitable for oral or parenteral administration.

16. The method of claim 12, wherein the pharmaceutical composition is in the form of a solution, a tablet, a capsule, or a transdermal delivery system.

17. The method of claim 12, wherein the pharmaceutical composition is in the form of an injectable solution.
